(19) United States
(12) Patent Application Publication
(10) Pub. No.: US 2004/0054006 A1
(43) Pub. Date: Mar. 18, 2004

(54) Use of creatine or creatine analogs for the prevention and treatment of Transmissible Spongiform Encephalopathies

(76) Inventors: Rima Kaddurah-Daouk, Belmont, MA (US); Yazan Mufti, Amman (JO)

Correspondence Address: LAHIVE & COCKFIELD, LLP., 28 STATE STREET, BOSTON, MA, 02109, US

(21) Appl. No.: 10/624,785

(22) Filed: Jul. 22, 2003

Related U.S. Application Data
(63) Continuation of application No. 09/602,466, filed on Jun. 23, 2000, now abandoned.
(60) Provisional application No. 60/141,025, filed on Jun. 25, 1999.

(51) Int. Cl. A61K 31/198

(52) U.S. Cl. 514/565

(57) ABSTRACT

The present invention relates to the use of creatine compounds including creatine, creatine phosphate or analogs of creatine, such as creatine-pyruvate, creatine ascorbate, cyclocreatine, 3 guanidinopropionic acid, guanidinoacetate, homocyclocreatine for preventing or treating TSE diseases of the nervous system. The Creatine compounds which can be used in the present method include (1) creatine, creatine phosphate and analogs of these compounds which can act as substrates or substrate analogs for creatine kinase; (2) molecules that mimic the biological activity of creatine (3) molecules that modulate the creatine kinase system.

### RELATED APPLICATIONS

This application claims priority to U.S. Provisional Patent Application Serial No. 60/141,025, entitled “Use of Creatine or Creatine Analogs for the Prevention and Treatment of Transmissible Spongiform Encephalopathies,” filed on Jun. 25, 1999.

Creatine and creatine compounds are discussed in related applications including U.S. patent application Ser. No. 08/853,174, filed on May 7, 1997; U.S. patent application Ser. No. 08/914,887, filed on Aug. 19, 1997; U.S. patent application Ser. No. 08/736,967, filed on Oct. 25, 1996; and U.S. patent application Ser. No. 09/285,395, filed on Apr. 2, 1999. The entire contents of each of these applications, including all references cited therein, and hereby expressly incorporated herein by reference.

### BACKGROUND OF THE INVENTION

The creatine kinase/creatine phosphate energy system is only one component of an elaborate energy-generating system found in nervous system cells such as, for example, neurons, oligodendrocytes and astrocytes. The components of the creatine energy system include the enzyme creatine kinase, the substrates creatine and creatine phosphate, and the transporter of creatine. The reaction catalyzed by creatine kinase is: MgADP+PCr⁼+H⁺MgATP⁼+Cr. Some of the functions associated with this system include efficient regeneration of energy in cells with fluctuating and high energy demands, energy transport to different parts of the cell, phosphoryl transfer activity, ion transport regulation, and involvement in signal transduction pathways.

Creatine is a compound which is naturally occurring and is found in mammalian brain and other excitable tissues, such as skeletal muscle, retina and heart. Its phosphorylated form, creatine phosphate, also is found in the same organs and is the product of the creatine kinase reaction utilizing creatine as a substrate. Creatine and creatine phosphate can be synthesized relatively easily and are believed to be non-toxic to mammals. Creatine, creatine phosphate and the enzymes that utilize them as substrates, e.g., the creatine kinases represent an efficient system for the rapid regeneration of energy. Kaddurah-Daouk et al. (WO 92/08456 published May 29, 1992 and WO 90/09192, published Aug. 23, 1990; U.S. Pat. No. 5,321,030; and U.S. Pat. No. 5,324,731) describe methods of inhibiting the growth, transformation and/or metastasis of mammalian cells using related compounds. Examples of compounds described by Kaddurah-Daouk et al. include cyclocreatine, b-guanidino propionic acid, homocyclocreatine, 1-carboxymethyl-2-iminohexahydropyrimidine, guanidino acetate and carbocreatine. These same inventors have also demonstrated the efficacy of such compounds for combating viral infections (U.S. Pat. No. 5,321,030). Elebaly in U.S. Pat. No. 5,091,404 discloses the use of cyclocreatine for restoring functionality in muscle tissue. Cohn in PCT publication No. WO94/16687 described a method for inhibiting the growth of several tumors using creatine and related compounds. Kaddurah-Daouk et. al.(WO 96/14063) reported on the neuroprotective effect of creatine compounds especially against neurodegenerative diseases such as, for example, Huntington's, Parkinson's, ALS, and Alzheimer's diseases.

The nervous system is an unresting assembly of cells that continually receives information, analyzes and perceives it and makes decisions. The principle cells of the nervous system are neurons and neuroglial cells. Neurons are the basic communicating units of the nervous system and possess dendrites, axons and synapses required for this role. Neuroglial cells consist of astrocytes, oligodendrocytes, ependymal cells, and microglial cells. Collectively, they are involved in the shelter and maintenance of neurons. The functions of astrocytes are incompletely understood but probably include the provision of biochemical and physical support and aid in insulation of the receptive surfaces of neurons. In addition to their activities in normal brain, they also react to CNS injury by glial scar formation. The principle function of the oligodendrocytes is the production and maintenance of CNS myelin. They contribute segments of myelin sheath to multiple axons. Rapid energy generation seems to be essential for the proper function and preservation of cells of the nervous system. Several neurodegenerative diseases are characterized by impaired energy production.

Transmissible Spongiform Encephalopathies (TSEs) are a group of neurodegenerative diseases characterized by the formation of spongiform change in the brain and believed to be a result of infectious proteinaceous particles referred to as prions (Cashman, Canadian Medical Association Journal, vol 157 (10) 1381-1385, 1997). The most common form of TSE is called scrapie and has been noted in goats and sheep for more than two hundred years. The Bovine spongiform encephalopathy (BSE) is a fatal brain disease of cattle similar in many ways to other TSE related diseases. BSE is characterized by progressive degeneration of the nervous system, and derives its name from the spongy appearance of cattle brain tissue examined microscopically upon death. Its more common name, “Mad Cow Disease” stems from the symptoms exhibited by afflicted cattle. BSE-afflicted cattle have difficulty walking, develop abnormal posture, loose weight and experience behavioral changes such as nervousness and aggression. The infectious particles in this disease are thought to be self replicating proteins called prions. Related diseases in humans are Kuru, Gerstmann-Straussler-Scheinker disease (GSSD), Creutzfeldt-Jakob disease (CJD), and a new variant of CJD called vCJD. An outbreak of BSE in the UK in the mid 1980s and early 1990s with possible transmission to humans has focused a lot of attention on this whole class of neurodegenerative diseases.

### SUMMARY OF THE INVENTION

The present invention pertains to methods of treating or preventing Transmissible Spongiform Encephalopathies (TSEs) and, preferably, Bovine Spongiform Encephalopathy (BSE), for example, in humans or in cattle. The method includes administering to the susceptible or afflicted subject an amount of a creatine compound or compounds which modulate one or more of the structural or functional components of the creatine kinase/phosphocreatine system sufficient to prevent, reduce or ameliorate the symptoms of the disease. Compounds which are effective for this purpose include, for example, the natural compound creatine and analogs of creatine. The compounds may advantageously be developed as a food supplement, medical foods or drugs.

The present invention also provides compositions containing creatine compounds in combination with a pharmaceutically acceptable carrier, and effective amounts of other agents which act on the nervous system, to prophylactically and/or therapeutically treat a subject suffering from a disease of the TSE. The present invention further pertains to methods of use of creatine compounds in combination with other agents which act on the nervous system for treating TSE diseases. The invention also relates to the use of creatine compounds in a food mix or as a dietary supplement for the treatment or prevention of TSEs in subjects, preferably, cattle.

### DETAILED DESCRIPTION

The methods of the present invention generally comprise administering to an individual afflicted with transmissible spongiform encephalopathies (TSES) (preferably, bovine spongiform encephalopathy (BSE)), an amount of a creatine compound or compounds which modulate one or more of the structural or functional components of the creatine kinase/phosphocreatine system sufficient to prevent, reduce or ameliorate symptoms of the disease.

The term “afflicted” includes both subjects suffering from the disorder as well as subjects at risk of contracting said disorder. Components of the system which can be modulated include the enzyme creatine kinase, the substrates creatine and creatine phosphate, and the transporter of creatine.

As used herein, the term “modulate” means to change, affect or interfere with the normal functioning of the component in the creatine kinase/phosphocreatine enzyme system.

Such compounds are predicted to preserve neuronal tissue by boosting up energy reserves in the brain and also by arresting mechanisms involved in the death of cells of the nervous system. Compounds which are particularly effective for this purpose include creatine, creatine phosphate, and analogs thereof which are described in detail below. The term “creatine compounds” will be used herein to include creatine, creatine phosphate, and compounds which are structurally similar to creatine or creatine phosphate, and analogs of creatine and creatine phosphate. The term “creatine compounds” also includes compounds which “mimic” the activity of creatine, creatine phosphate or creatine analogs. The term “mimics” is intended to include compounds which may not be structurally similar to creatine but mimic the therapeutic activity of creatine, creatine phosphate or structurally similar compounds. Also the term creatine compound includes “modulators of the creatine kinase system,” e.g., compounds which modulate the activity of the enzyme, or the activity of the transporter of creatine or the ability of other proteins or enzymes or lipids to interact with the system.

The language “treating diseases of TSEs” includes prevention of the disease, amelioration and/or arrest of a preexisting disease, and the elimination of a preexisting disease. The creatine analogs described herein have both curative and prophylactic effects on disease development and progression.

The language “therapeutically effective amount” includes the amount of the creatine compound sufficient to prevent onset of TSEs diseases or significantly reduce progression of such diseases in the subject being treated. A therapeutically effective amount can be determined on an individual basis and will be based, at least in part, on consideration of the severity of the symptoms to be treated and the activity of the specific analog selected if an analog is being used. Further, the effective amounts of the creatine compound may vary according to the age, sex and weight of the subject being treated. Thus, a therapeutically effective amount of the creatine compound can be determined by one of ordinary skill in the art employing such factors as described above using no more than routine experimentation in clinical management.

The language “pharmaceutically acceptable carrier” includes substances capable of being coadministered with the creatine compound and which allows the active ingredient to perform its intended function of preventing, ameliorating, arresting, or eliminating a disease(s) of TSEs. Examples of such carriers include sugars, solvents, dispersion media, adjuvants, delay agents, food preparations and the like. The use of such media and agents for pharmaceutically active substances is well known in the art. Any conventional media and agent compatible with the creatine compound may be used within this invention.

The term “pharmaceutically acceptable salt” is intended to include art-recognized pharmaceutically acceptable salts. Typically these salts are capable of being hydrolyzed under physiological conditions. Examples of such salts include sodium, potassium and hemisulfate. The term further is intended to include lower hydrocarbon groups capable of being hydrolyzed under physiological conditions, i.e. groups which esterify the carboxyl moiety, e.g. methyl, ethyl and propyl.

The term “subject” includes living organisms susceptible to having TSEs e.g. mammals. Examples of subjects include humans, cattle, dogs, cats, horses, goats, rats and mice. The term “subject” further is intended to include transgenic species.

The language “transmissible spongiform encephalopathies” or “TSEs” includes diseases of the nervous system with spongy pathology and prion involvement whose onset, amelioration, arrest, or elimination may be effectuated by the creatine compounds described herein. Examples of TSEs include Mad Cow Disease BSE and scrapies.

Human Transmissible Spongiform Encephalopathies (TSEs)

TSEs are a group of neurodegenerative diseases characterized by the formation of spongiform change in the brain and believed to be a result of infectious proteinaceous particles referred to as prions (Cashman, Canadian Medical Association Journal, vol. 157 (10) 1381-1385, 1997). Many of the TSE diseases that we know about have only appeared in the last decade like Bovine spongiform encephalopathy (BSE) in cattle. Others such as scrapie in sheep and Creutzfeldt-Jakob disease (CJD) in humans have been known for a long time. The effects of the various strains of TSE are all fairly similar, causing gradual dementia in the afflicted. This dementia is believed to be due to the infectious agent found in the brains that gradually build up into plaques. These deposits then have a negative effect on the surrounding tissue resulting in nerve cell death, generally in the cerebrum and cerebellum areas of the brain. This gives the brain the characteristic spongy appearance which is clearly visible postmortem. (Brown, J. Amer. Med. Assoc., 278(12):1008-1011, 1997).

These spongiform signs can only be seen after death. Earlier signs of degeneration of the brain are manifested by symptoms like loss of muscle control and spasticity, rigidity and tremor. Occasionally cortical blindness, and motor neuron involvement resulting in weakness, fasciculation and amyotrophy occur.

Prions

Although there are several theories of what causes transmissible spongiform encephalopathies (TSEs), scientific evidence suggests that they are unique proteins that, unlike other infectious agents, contains no genetic material. The agent, originally described as self replicating protein, has been most recently described as prion, which is an aberrant conformation of normal proteins. Prions resemble normal cellular proteins except for minor but crucial changes in structure. Their unique infectious ability is apparently derived from their ability to cause a normal protein molecule to change shape upon contact, perhaps by inducing it to refold itself into the aberrant confirmation. Molecules so formed go on to do the same to other normal proteins, thus creating further aberrant replicas from an originally normal protein (Prusiner, Scientific America vol. 272, 48-57, 1995, Prusiner, Archives of Neurology vol. 50, 1129-1153, 1993.

Prusiner and colleagues purified and partially sequenced a protease resistant protein fragment from Scrapie infected hamster brain that copurified with Scrapie activity, allowing for the molecular cloning and sequencing of its cognate cDNA. This infectious protein was found to be encoded by an ancient highly conserved host gene (Basler et. al., Cell, vol. 46, 417-428, 1986). It was interesting to find that the same nucleic acid and amino acid sequence could give rise to two proteins with very different properties; in normal cellular isoform (PrP sup C) expressed a normal brain, readily soluble and protease sensitive protein. An abnormal Scrapie associated isoform (PrP sup Sc) accumulated in TSE brain, is poorly soluble, partially protease resistant and associated with infectious activity. Later work demonstrated that these two isoforms are different in their conformation (Prusiner, Curr.Top. Microb. Immunol. vol. 207, 1-17, 1996). It seems that infectivity is a result of this conformational change.

Scrapie

Scrapie is the most common form of TSE. It was first recognized in 1730 and is a disease of sheep, so named after the habit of affected animals to repeatedly scrape themselves against objects. Eventually the animals become unsteady on their feet, uninterested in their surroundings and stopped feeding. Examination of the brains of affected sheep after death show the characteristic spongiform tissue.

Bovine Spongiform Encephalopathy (BSE)“Mad Cow Disease”

The Bovine spongiform encephalopathy (BSE) discovered in 1986, is a fatal brain disease of cattle characterized by progressive degeneration of the nervous system. BSE derives its name from the spongy appearance of cattle brain tissue examined microscopically upon death. It's more common name, “Mad-Cow Disease” stems from the symptoms exhibited by afflicted cattle. BSE-afflicted cattle have difficulty walking, develop abnormal posture, loose weight and experience behavioral changes such as nervousness and aggression. (For references see Nutrition Reviews vol. 54 (7) 208-210, 1996; Carr, Nature, vol. 380, 273-274, 1996).

Although there are several theories of what causes BSE, It is suspected that the BSE epidemic in the United Kingdom which surfaced from the mid 1980's to the early 1990's (over 160,000 confirmed cases reported and over one million suspected infected cattle, Anderson et. al., Nature vol. 382, 779-788, 1996) was caused by the use of protein feed supplements made from meat and bonemeal of carcasses of Scrapie infected sheep. The other theory suggests that BSE previously existed at a low level in cattle but was amplified by the practice of including meat and bonemeal of cattle as protein supplement in cattle feed. This practice of feeding ruminant materials to cattle was later abolished resulting in decreased cases of BSE. The Scrapie agent which is believed to be a prion in nature has been passed in cell cultures but has not been cultured efficiently in any system. The agent is experimentally transmissible from sheep to sheep and goats and into a range of mammalian species including cattle (Hourrigan J. Am. Vet. Med. Assoc. vol. 196, 1678-1679, 1990; Gibbs et. al., Lancet vol. 335, 1275, 1990; Clark et. al., Am.J. Vet. Res. vol. 56, 606-612, 1995; Cutlip et. al., J. Infect. Dis. vol. 169, 814-820, 1994). The experimentally transmitted cattle disease via Scrapie injection is clinically and pathologically different from naturally occurring BSE.

Human TSEs

In the mid 1950's Dr. Carleton Gajdusek reported on a tribe in New Guinea which practiced cannibalism since the stone age and had an increased susceptibility to the TSE known as Kuru disease. Kuru results in a progressive ataxic syndrome with late dementia and other neurological impairments. There is loss of neurons, proliferation of astrocytes and microglial cells, and spongiform change which is microscopic vacuoles in the brain parenchyma. There is also an accumulation of an abnormal protease-resistant protein in the brain plaques later found to be PrP-sup Sc which is a human form of Scrapie.

Creutzfeldt-Jakob disease (CJD was first described in the 1920's and is the most common spongy form of encephalopathy in humans. CJD occurs worldwide, is relatively rare and appears to occur randomly at the rate of one person per million. It may be sporadic (random cases with no known cause, acquired from environmental sources (contaminated pituitary hormones or dura mater grafts from animals), or familial (due to mutations in a gene on chromosome 20). The onset of the disease occurs at the age of sixty although cases of younger onset have been documented. Like BSE, CJD is fatal. Cerebral symptoms occur late in two thirds of the CJD cases with dementing onset.

Although no cases of CJD have been directly linked to beef consumption some recent variant forms of CJD has been suggested to be linked to BSE (Will lancet, vol. 347, 921-925, 1996; Lasmezas Nature, vol. 381, 743-744, 1996). The transmission of BSE to humans through feeding on infected cattle is uncertain at this point. Several forms of spongy form encephalopathies have been experimentally transmitted to mice, pigs, goats, sheep, goats by parental injection of infected brain homogenates (Collee and Bradley Lancet, vol. 349, 636-641, 1997). With the oral route it is transmissible in some cases with very high challenge doses. Also transgenic mice are experimented on for further characterization of the disease (Collinge et. al., Nature vol. 378, 779-782, 1995). It seems that humans appear to be the one species susceptible to interspecies transfer of the BSE agent.

Prion Diseases: Diagnosis

Up until recently, confirmation of the TSE diseases was done by microscopic examination of brain tissue post death. Mutations in the protein coding sequence of the prion protein gene have now been identified. Assays have been developed with some success for the detection of prion infections. Recently a test of the cerebrospinal fluid has been advanced to assist CJD diagnosis, based on the appearance of neuronal cell signaling protein 14-3-3 (Hsich, New Eng.J.of Med. vol. 335, 924-930, 1996; Collinge New England Journal of Medicine, vol. 335, 963-965, 1996; Lee and Herrington, Lancet, vol. 348, 887, 1996; Will et. al., Lancet, vol. 348, 955, 1996). This protein is detectable in disorders with rapid rate of neuronal loss such as CJD, viral encephalitis, cerebral infarct, and some cases of Alzheimer's. Unfortunately for early detection of the disease prior to symptom development this test is not optimal. Also a more sensitive and specific diagnostic test is not available.

Therapy for TSEs

There does not seem to be imminent prospects for therapy for the diseases described above. Better understanding at the molecular level the genesis and progression of these diseases is needed to unravel new approaches for therapy.

Creatine Kinase Isoenzymes in the Brain

Cells require energy to survive and to carry out the multitude of tasks that characterize biological activity. Cellular energy demand and supply are generally balanced and tightly regulated for economy and efficiency of energy use. Creatine kinase plays a key role in the energy metabolism of cells with intermittently high and fluctuating energy requirements such as skeletal and cardiac muscle, brain and neural tissues, including, for example, the retina, spermatozoa and electrocytes. As stated above, the enzyme catalyzes the reversible transfer of the phosphoryl group from creatine phosphate to ADP, to generate ATP. There are multi-isoforms of creatine kinase (CK) which include muscle (CK-MM), brain (CK-BB) and mitochondrial (CK-Mia, CK-Mib) isoforms.

Experimental data suggest that CK is located near the sites in cells where energy generation occurs; e.g., where force generation by motor proteins takes place, next to ion pumps and transporters in membranes and where other ATP-dependent processes take place. It seems to play a complex multi-faceted role in cellular energy homeostasis. The creatine kinase system is involved in energy buffering/energy transport activities. It also is involved in regulating ADP and ATP levels intracellularly as well as ADP/ATP ratios. Proton buffering and production of inorganic phosphate are important parts of the system.

In the brain, this creatine kinase system is quite active. Regional variations in CK activity with comparably high levels in cerebellum were reported in studies using native isoenzyme electrophoresis, or enzymatic CK activity measurements in either tissue extracts or cultured brain cells. Chandler et al. Stroke, 19: 251-255 (1988), Maker et al. Exp. Neurol., 38: 295-300 (1973), Manos et al. J. Neurol. Chem., 56: 2101-2107 (1991). In particular, the molecular layer of the cerebellar cortex contains high levels of CK activity (Maker et al. id. (1973) Kahn Histochem., 48: 29-32 (1976) consistent with the recent 3′P-NMR findings which indicate that gray matter shows a higher flux through the CK reaction and higher creatine phosphate concentrations as compared to white matter (Cadoux-Hudson et al. FASEBJ., 3: 2660-2666 (1989), but also high levels of CK activity were shown in cultured oligodendrocytes (Manos et al. id. (1991), Molloy et al. J. Neurochem., 59: 1925-1932 (1992), typical glial cells of the white matter. The brain CK isoenzyme CK-BB is the major isoform found in the brain. Lower amounts of muscle creatine kinase (CK-MM) and mitochondrial creatine kinase (CK-Mi) are found.

Localization and Function of CK Isoenzymes in Different Cells of the Nervous System

Brain CK (CK-BB) is found in all layers of the cerebellar cortex as well as in deeper nuclei of the cerebellum. It is most abundant in Bergmann glial cells (BGC) and astroglial cells, but is also found in basket cells and neurons in the deeper nuclei. Hemmer et al., Eur. J. Neuroscience, 6: 538-549 (1994), Hemmer et al. Dev. Neuroscience, 15: 3-5 (1993). The BGC is a specialized type of astroglial cell. It provides the migratory pathway for granule cell migration from the external to the internal granule cell layer during cerebellar development. Another main function of these cells is the proposed ATP-dependent spatial buffering of potassium ions released during the electrical activity of neurons (Newman et al. Trends Neuroscience, 8: 156-159 (1985), Reichenbach, Acad. Sci New York, (1991), pp. 272-286. Hence, CK-BB seems to be providing energy (ATP) for migration as well as K⁺ buffering through regulation of the Na⁺/K⁺ ATPase. The presence of CK-BB in astrocytes (Manos et al. id. 1991, Hemmer et al. id. 1994, Hemmer et al. id. 1993) may be related to the energy requirements of these cells for metabolic interactions with neurons; e.g., tricarboxylic acid cycle (TCA) metabolite and neurotransmitter trafficking. Hertz, Can J. Physiol. Pharmacol., 70: 5145-5157 (1991).

The Purkinje neurons of the cerebellum play a very important role in brain function. They receive excitatory input from parallel fibers and climbing fibers, they represent the sole neuronal output structures of the cerebellar cortex. Calcium mediated depolarizations in Purkinje cell dendrites are thought to play a central role in the mechanism of cerebellar motoric learning. Ito Corr. Opin. Neurobiol., 1: 616-620 (1991). High levels of muscle CK (CK-MM) were found in Purkinje neurons. Hemmer et al. id. (1994), Hemmer et al., id. (1993). There is strong evidence to support that CK-MM is directly or indirectly coupled to energetic processes needed for Ca⁺⁺ homeostasis or to cellular processes triggered by this second messenger.

The glomerular structures of the cerebellum contain high levels of CK-BB and mitochondrial CK (CK-Mi). Large amounts of energy are needed in these structures for restoration of potassium ion gradients partially broken down during neuronal excitation as well as for metabolic and neurotransmitter trafficking between glial cells and neurons. Hertz et al., id. (1991). The presence of CK in these structures may be an indication that part of the energy consumed in these giant complexes might be supported by the creatine kinase system.

In neurons, CK-BB is found in association with synaptic vesicles (Friedhoff and Lemer, Life Sci., 20: 867-872 (1977) as well as with plasma membranes (Lim et al., J. Neurochem., 41: 1177-1182 (1983)).

There is evidence to suggest that CK is bound to synaptic vesicles and to the plasma membrane in neurons may be involved in neurotransmitter release as well as in the maintenance of membrane potentials and the restoration of ion gradients before and after stimulation. This is consistent with the fact that high energy turnover and concomitantly high CK concentrations have been found in those regions of the brain that are rich in synaptic connections; e.g., in the molecular layer of the cerebellum, in the glomerular structures of the granule layer and also in the hippocampus. The observation that a rise in CK levels observed in a fraction of brain containing nerve endings and synapses, parallels the neonatal increase in Na⁺/K⁺ ATPase is also suggestive that higher levels of creatine phosphates and CK are characteristic of regions in which energy expenditure for processes such as ion pumping are large. Erecinska and Silver, J. Cerebr. Blood Flow and Metabolism, 9: 2-19 (1989). In addition, protein phosphorylation which plays an important role in brain function is also through to consume a sizable fraction of the total energy available in those cells (Erecinska and Silver, id. 1989). Finally, CK, together with nerve-specific enolase belongs to a group of proteins known as slow component b (SCb). These proteins are synthesized in neuronal cell body and are directed by axonal transport to the axonal extremities. Brady and Lasek, Cell, 23: 515-523 (1981), Oblinger et al., J. Neurol., 7: 433-462 (1987) The question of whether CK participates in the actual energetics of axonal transport remains to be answered.

In conclusion, the CK system plays a key role in the energetics of the adult brain. This is supported by ³¹P NMR magnetization transfer measurements showing that the pseudo first order rate constant of the CK reaction in the direction of ATP synthesis as well as CK flux correlate with brain activity which is measured by EEG as well as by the amount of deoxyglucose phosphate formed in the brain after administration of deoxyglucose. The present inventors describes the use of creatine compounds for the treatment of transmissible spongiform encephalopathies (TSEs) by modulating the activity of the creatine kinase/creatine phosphate pathway.

The Role of Creatine Kinase in Treating Transmissible Spongiform Encephalopathies (TSEs)

The mechanisms by which nerve cell metabolites are normally directed to specific cell tasks is poorly understood. It is thought that nerve cells, like other cells, regulate the rate of energy production in response to demand. The creatine kinase system is active in many cells of the nervous system and is thought to play a key role in the allocation of high energy phosphate to many diverse neurological processes, such as neurotransmitter biosynthesis, electrolyte flux and synaptic communication. Neurological function requires significant energy and creatine kinase appears to play an important role in controlling the flow of energy inside specialized exitable cells such as neurons. The induction of creatine kinase, the BB isozyme and the brain mitochondrial creatine kinase in particular, results in the generation of a high energy state which could sustain and preserve neuronal function and survival.

The components of the creatine kinase/phosphocreatine system include the enzyme creatine kinase, the substrates creatine and creatine phosphate, and the transporter of creatine. Some of the functions associated with this system include efficient regeneration of energy in cells with fluctuating and high energy demand, phosphoryl transfer activity, ion transport regulation, cytoskeletal association, nucleotide pool preservation, proton buffering, and involvement in signal transduction pathways. The creatine kinase/phosphocreatine system has been shown to be active in neurons, astrocytes, oligodendrocytes, and Schwann cells. The activity of the enzyme has been shown to be up-regulated during regeneration and down-regulated in degenerative states, and aberrant in mitochondrial diseases.

Many diseases of the nervous system are thought to be associated with abnormalities in an energy state which could result in imbalanced ion transport neurotransmitter release and result in cell death. It has been reported that defects in mitochondrial respiration enzymes and glycolytic enzymes may cause impairment of cell function and could be associated with neurodegenerative processes.

Without wishing to be bound by theory, it is thought that modulating the creatine kinase activity would modulate energy flow and affect cell function and survival. An activated energy state should enable cells of the nervous system to withstand insult such as infection with prions.

Ingestion of creatine analogs has been shown to result in replacement of tissue phosphocreatine pools by synthetic phosphagens with different kinetic and thermodynamic properties. This results in subtle changes of intracellular energy metabolism, including the increase of total reserves of high energy phosphate (see refs. Roberts, J. J. and J. B. Walker, Arch Biochem. Biophys 220(2): 563-571 (1983)). The replacement of phosphocreatine pools with slower acting synthetic phosphagens, such as creatine analogs might benefit neurological disorders by providing a longer lasting source of energy. One such analog, cyclocreatine (1-carboxymethyl-2-aminoimidazolidine) modifies the flow of energy of cells in stress and may interfere with ATP utilization at sites of cellular work.

Creatine Compounds Useful for Treating TSEs

Creatine compounds useful in the present invention include compounds which modulate one or more of the structural or functional components of the creatine kinase/phosphocreatine system. Compounds which are effective for this purpose include creatine, creatine phosphate and analogs thereof, compounds which mimic their activity, and salts of these compounds as defined above. Exemplary creatine compounds are described below.

Creatine (also known as N-(aminoiminomethyl)-N-methylglycine; methylglycosamine or N-methyl-guanido acetic acid) is a well-known substance. (See, The Merck Index, Eleventh Edition, No. 2570 (1989). It could come in different salt forms and hydration states.

Creatine is phosphorylated chemically or enzymatically by creatine kinase to generate creatine phosphate, which also is well-known (see, The Merck Index, No. 7315). Both creatine and creatine phosphate (phosphocreatine) can be extracted from animal tissue or synthesized chemically. Both are commercially available.

Cyclocreatine is an essentially planar cyclic analog of creatine. Although cyclocreatine is structurally similar to creatine, the two compounds are distinguishable both kinetically and thermodynamically. Cyclocreatine is phosphorylated efficiently by creatine kinase in the forward reaction both in vitro and in vivo. Rowley, G. L., J. Am. Chem. Soc. 93: 5542-5551 (1971); McLaughlin, A. C. et. al., J. Biol. Chem. 247, 4382-4388 (1972).

The phosphorylated compound phosphocyclocreatine is structurally similar to phosphocreatine; however, the phosphorous-nitrogen (P—N) bond of cyclocreatine phosphate is more stable than that of phosphocreatine. LoPresti, P. and M. Cohn, Biochem. Biophys. Acta 998: 317-320 (1989); Annesley, T. M. and J. B. Walker, J. Biol. Chem. 253; 8120-8125, (1978); Annesley, T. M. and J. B. Walker, Biochem. Biophys. Res. Commun. 74: 185-190 (1977).

3-Guanidinopropionic acid (3-GPA) is an endogenous metabolite found in animals and humans (Hiraga et.al., J. of Chromatography vol 342, 269-275, 1985; Watanabe et.al., Guanidines edited by Mori et.al., Plenum, N.Y., 49-58, 1983). The compound is available from Sigma chemicals and is an extensively studied analog of creatine.

Guanidino acetate is yet another analog of creatine and is a precursor of creatine in its biosynthetic pathway. Guanidino benzoates such as 4-guanidino benzoate are also creatine compounds of use for the present invention

Creatine analogs and other agents which act to interfere with the activity of creatine biosynthetic enzymes or with the creatine transporter are useful in the present method of treating TSEs. In the nervous system, there are many possible intracellular, as well as extracellular, sites for the action of compounds that inhibit, increase, or otherwise modify, energy generation through brain creatine kinase and/or other enzymes which are associated with it. Thus the effects of such compounds can be direct or indirect, operating by mechanisms including, but not limited to, influencing the uptake or biosynthesis of creatine, the function of the creatine phosphate shuttle, enzyme activity, or the activity of associated enzymes, or altering the levels of substrates or products of a reaction to alter the velocity of the reaction.

Substances known or believed to modify energy production through the creatine kinase/phosphocreatine system which can be used in the present method are described below. Exemplary compounds are shown in Tables 1 and 2.

It will be possible to modify the substances described below to produce analogs which have enhanced characteristics, such as greater specificity for the enzyme, enhanced stability, enhanced uptake into cells, or better binding activity.

Compounds which modify the structure or function of the creatine kinase/creatine phosphate system directly or indirectly are useful in preventing and/or treating TSEs.

Molecules that regulate the transporter of creatine, or the association of creatine kinase with other protein or lipid molecules in the membrane, the substrates concentration creatine and creatine phosphate also are useful in preventing and/or treating TSEs.

Compounds which are useful in the present invention can be substrates, enzyme activity modifiers or substrate analogs of creatine kinase. In addition, modulators of the enzymes that work in conjunction with creatine kinase now can be designed and used, individually, in combination or in addition to creatine compounds. Combinations of creatine compounds with other supplements or other drugs is proposed.

The pathways of biosynthesis and metabolism of creatine and creatine phosphate can be targeted in selecting and designing compounds which may modify energy production or high energy phosphoryl transfer through the creatine kinase system.

Compounds targeted to specific steps may rely on structural analogies with either creatine or its precursors. Novel creatine analogs differing from creatine by substitution, chain extension, and/or cyclization may be designed. The substrates of multisubstrate enzymes may be covalently linked, or analogs which mimic portions of the different substrates may be designed. Non-hydrolyzable phosphorylated analogs can also be designed to mimic creatine phosphate without sustaining ATP production.

A number of creatine and creatine phosphate analogs have been previously described in the literature or can be readily synthesized. Examples are these shown in Table 1 and Table 2. Some of them are slow substrates for creatine kinase.

TABLE 1
CREATINE ANALOGS

| | | |
|---|---|---|
| 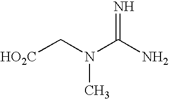 | 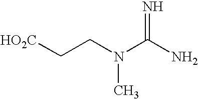 | 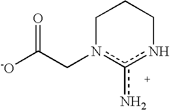 |
| 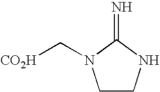 | 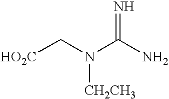 | 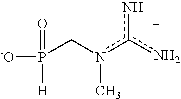 |
| 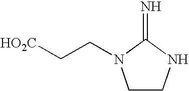 | 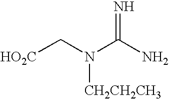 | 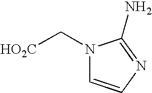 |
| 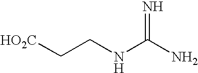 | 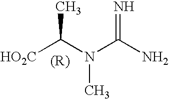 | 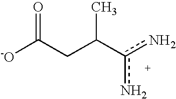 |
| 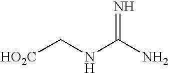 | 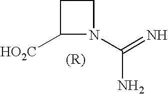 | |
| 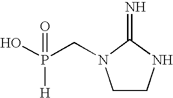 | 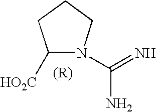 | |
| 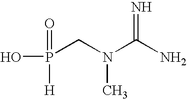 | | |

TABLE 2
CREATINE PHOSPHATE ANALOGS

| | |
|---|---|
| 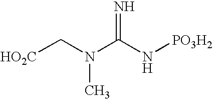 | 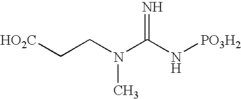 |
| 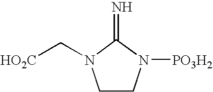 | 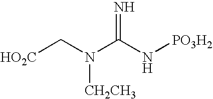 |
| 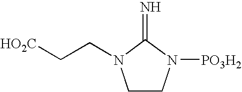 | 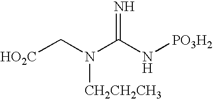 |
| 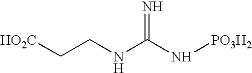 | 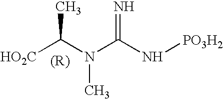 |
| 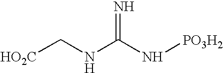 | 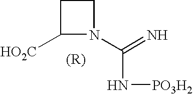 |
| 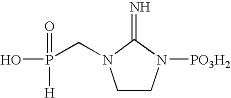 | 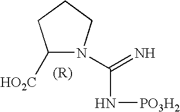 |
| 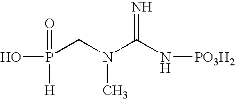 | 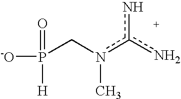 |
| 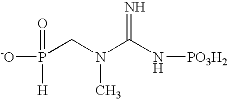 | |

Tables 1 and 2 illustrate the structures of creatine, cyclocreatine (1-carboxymethyl-2-iminoimidazolidine), N-phosphorocreatine (N-phosphoryl creatine), cyclocreatine phosphate (3-phosphoryl-1-carboxymethyl-2-iminoimidazolidine) and other compounds. In addition, 1-carboxymethyl-2-aminoimidazole, 1-carboxymethyl-2 2-iminomethylimidazolidine, 1-carboxyethyl-2-iminoimidazolidine, N-ethyl-N-amidinoglycine and b-guanidinopropionic acid are believed to be effective.

Cyclocreatine (1-carboxymethyl-2-iminoimidazolidine) is an example of a class of substrate analogs of creatine kinase, which can be phosphorylated by creatine kinase and which are believed to be active.

A class of creatine kinase targeted compounds are bi-substrate analogs comprising an adenosine-like moiety linked via a modifiable bridge to a creatine link moiety (i.e., creatine or a creatine analog). Such compounds are expected to bind with greater affinity than the sum of the binding interaction of each individual substrate (e.g., creatine and ATP). The modifiable bridge linking an adenosine-like moiety at the 5′-carbon to a creatine like moiety can be a carbonyl group, alkyl (a branched or straight chain hydrocarbon group having one or more carbon atoms), or substituted alkyl group (an alkyl group bearing one or more functionalities, including but not limited to unsaturation, heteroatom-substituents, carboxylic and inorganic acid derivatives, and electrophilic moieties).

The attachment of creatine covalently to other molecules is an easy way to generate creatine compounds. Examples are molecules that have already been synthesized such as creatine-pyruvate and creatine ascorbate. Amino acid like molecules could be attached to creatine as well as a diverse group of molecules. These are prototypes of creatine compounds with are useful for the present invention.

N-phosphorocreatine analogs also can be designed which bear non-transferable moieties which mimic the N-phosphoryl group. These cannot sustain ATP production.

Some currently preferred creatine compounds of this invention are those encompassed by the general formula I:

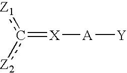

and pharmaceutically acceptable salts thereof, wherein:

a) Y is selected from the group consisting of: —CO₂H—NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;

b) A is selected from the group consisting of: C, CH, C₁-C₅alkyl, C₂-C₅alkenyl, C₂-C₅alkynyl, and C₁-C₅alkoyl chain, each having 0-2 substituents which are selected independently from the group consisting of:

1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;

2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and

3) —NH—M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;

c) X is selected from the group consisting of NR₁, CHR₁, CR₁, O and S, wherein R₁ is selected from the group consisting of:

1) hydrogen;

2) K where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;

3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;

4) a C₅-C₉ a-amino-w-methyl-w-adenosylcarboxylic acid attached via the w-methyl carbon;

5) 2 C₅-C₉ a-amino-w-aza-w-methyl-w-adenosylcarboxylic acid attached via the w-methyl carbon; and

6) a C₅-C₉ a-amino-w-thia-w-methyl-w-adenosylcarboxylic acid attached via the w-methyl carbon;

d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:

1) hydrogen;

2) K, where K is selected from the group consisting of: C₁-C₆ straight alkyl; C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;

3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;

4) 2 C₄-C₈ a-amino-carboxylic acid attached via the w-carbon;

5) B, wherein B is selected from the group consisting of: —CO₂H—NHOH, —SO₃H, —NO₂, OP(═O)(OH)(OJ) and —P(═O)(OH)(OJ), wherein J is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;

6) —D—E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙNMP, where n is 0-2 and NMP is ribonucleotide monophosphate connected via the 5′-phosphate, 3′-phosphate or the aromatic ring of the base; —[P(═O)(OCH₃)(O)]ₘ—Q, where m is 0-3 and Q is a ribonucleoside connected via the ribose or the aromatic ring of the base; —[P(═O)(OH)(CH₂)]ₘ—Q, where m is 0-3 and Q is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and

7) —E, wherein E is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ—Q, where m is 0-3 and Q is a ribonucleoside connected via the ribose or the aromatic ring of the base; —[P(═O)(OH)(CH₂)]ₘ—Q, where m is 0-3 and Q is a ribonucleoside connected via the ribose or the aromatic ring of the base; and an aryl group containing 0-3 substituents chose independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E is aryl, E may be connected by an amide linkage;

e) if R₁ and at least one R₂ group are present, R₁ may be connected by a single or double bond to an R₂ group to form a cycle of 5 to 7 members;

f) if two R₂ groups are present, they may be connected by a single or a double bond to form a cycle of 4 to 7 members; and

g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

Creatine, creatine phosphate and many creatine analogs are commercially available. Additionally, analogs of creatine may be synthesized using conventional techniques. For example, creatine can be used as the starting material for synthesizing at least some of the analogs encompassed by formula I. Appropriate synthesis reagents, e.g. alkylating, alkenylating or alkynylating agents may be used to attach the respective groups to target sites. Alternatively, reagents capable of inserting spacer groups may be used to alter the creatine structure. Sites other than the target site are protected using conventional protecting groups while the desired sites are being targeted by synthetic reagents.

If the creatine analog contains a ring structure, then the analog may be synthesized in a manner analogous to that described for cyclocreatine (Wang, T., J. Org, Chem, 39:3591-3594 (1974)). The various other substituent groups may be introduced before or after the ring is formed.

Many creatine analogs have been previously synthesized and described (Rowley et al., J. Am. Chem. Soc. 93:5542-5551 (1971); McLaughlin et al., J. Biol. Chem. 247:4382-4388 (1972); Nguyen, A. C. K., “Synthesis and enzyme studies using creatine analogs”, Thesis, Dept. of Pharmaceutical Chemistry, Univ. Calif., San Francisco (1983); Lowe et al., J. Biol. Chem. 225:3944-3951 (1980); Roberts et al., J. Biol. Chem. 260:13502-13508 (1985); Roberts et al., Arch. Biochem. Biophys. 220:563-571 (1983), and Griffiths et al., J. Biol. Chem. 251:2049-2054 (1976)). The contents of all of the forementioned references are expressly incorporated by reference. Further to the forementioned references, Kaddurah-Daouk et al. (WO92/08456; WO90/09192; U.S. Pat. No. 5,324,731; U.S. Pat. No. 5,321,030) also provide citations for the synthesis of a plurality of creatine analogs. The contents of all the aforementioned references and patents are incorporated herein by reference.

Creatine compounds which currently are available or have been synthesized include, for example, creatine, b-guanidinopropionic acid, guanidinoacetic acid, creatine phosphate disodium salt, cyclocreatine, homocyclocreatine, phosphinic creatine, homocreatine, ethylcreatine, cyclocreatine phosphate dilithium salt and guanidinoacetic acid phosphate disodium salt, among others.

Creatine phosphate compounds also can be synthesized chemically or enzymatically. The chemical synthesis is well known. Annesley, T. M. Walker, J. B., Biochem. Biophys. Res. Commun., (1977), 74, 185-190; Cramer, F., Scheiffele, E., Vollmar, A., Chem. Ber., (1962), 95, 1670-1682.

Salts of the products may be exchanged to other salts using standard protocols. The enzymatic synthesis utilizes the creatine kinase enzyme, which is commercially available, to phosphorylate the creatine compounds. ATP is required by creatine kinase for phosphorylation, hence it needs to be continuously replenished to drive the reaction forward. It is necessary to couple the creatine kinase reaction to another reaction that generates ATP to drive it forward. The purity of the resulting compounds can be confirmed using known analytical techniques including ¹H NMR, ¹³CNMR Spectra, Thin layer chromatography, HPLC and elemental analysis.

Modes of Administration

The creatine compound can be administered to the afflicted individual alone or in combination with another creatine analog or other agent. The other agents could be approved therapies, supplements that protect the nervous system, vitamins, antioxidants, sugars and nutrients among others. The creatine compounds can be administered as pharmaceutically acceptable salts in a pharmaceutically acceptable carrier. The compound may be administered to the subject by a variety of routes, including, but not necessarily limited to, oral (dietary), transdermal, or parenteral (e.g., subcutaneous, intramuscular, intravenous injection, bolus or continuous infusion) routes of administration, for example. An effective amount (i.e., one that is sufficient to produce the desired effect in an individual) of a composition comprising a creatine analog is administered to the individual. The actual amount of drug to be administered will depend on factors such as the size and age of the individual, in addition to the severity of symptoms, other medical conditions and the desired aim of treatment.

Previous studies have described the administration and efficacy of creatine compounds in vivo. Creatine monohydrate is taken by athletes and body builders at high amounts ranging from 2-30 gms per day. Creatine phosphate has been administered to patients with cardiac diseases by intravenous injection. Up to 8 grams/day were administered with no adverse side effects. The efficacy of selected creatine kinase substrate analogs to sustain ATP levels or delay rigor during ischemic episodes in muscle has been investigated. On one study, cyclocreatine was fed to mice, rats and chicks, and appeared to be well-tolerated in these animals. Newly hatched chicks were fed a diet containing 1% cyclocreatine. In the presence of antibiotics, the chicks tolerated 1% cyclocreatine without significant mortality, although the chicks grew more slowly than control chicks (Griffiths, G. R. and J. B. Walker, J. Biol. Chem. 251(7): 2049-2054 (1976)). In another study, mice were fed a diet containing 1% cyclocreatine for 10 days (Annesley, T. M. and J. B. Walker, J. Biol. Chem. 253(22): 8120-8125 (1978)). Cyclocreatine has been feed to mice at up to 1% of their diet for 2 weeks or for over 4 weeks without gross adverse effects. Lillie et al., Cancer Res., 53: 3172-3178 (1993). Feeding animals cyclocreatine (e.g., 1% dietary) has been shown to lead to accumulation of cyclocreatine in different organs in mM concentrations. For example, cyclocreatine was reported to be taken up by muscle, heart and brain in rats receiving dietary 1% cyclocreatine. Griffiths, G. R. and J. B. Walker, J. Biol. Chem. 251(7): 2049-2054 (1976). As shown previously, antiviral activity of cyclocreatine is observed on administering 1% dietary cyclocreatine. Many of the above-referenced studies show that creatine analogs are been shown to be capable of crossing the blood-brain barrier. Creatine as 1%-3% of the diet was shown to have beneficial effects on Huntington's disease.

The creatine compound can be formulated according to the selected route of administration (e.g., powder, tablet, capsule, transdermal patch, implantable capsule, solution, emulsion). An appropriate composition comprising a creatine analog can be prepared in a physiologically acceptable vehicle or carrier. For example, a composition in tablet form can include one or more additives such as a filler (e.g., lactose), a binder (e.g., gelatin, carboxymethylcellulose, gum arabic), a flavoring agent, a coloring agent, or coating material as desired. For solutions or emulsions in general, carriers may include aqueous or alcoholic/aqueous solutions, emulsions or suspensions, including saline and buffered media. Parenteral vehicles can include sodium chloride, solution, Ringer's dextrose, dextrose and sodium chloride, lactated Ringer's or fixed oils. In addition, intravenous vehicles can include fluid and nutrient replenishers, and electrolyte replenishers, such as those based on Ringer's dextrose. Preservatives and other additives can also be present. For example, antimicrobial, antioxidant, chelating agents, and inert gases can be added. (See, generally, Remington's Pharmaceutical Sciences, 16th Edition, Mack, Ed., 1980). Creatine or analogs could be added to the diet of cattle as a certain percentage of the diet to prevent the occurance of BSE.

The term “administration” is intended to include routes of administration which allow the creatine compounds to perform their intended function(s) of preventing, ameliorating, arresting, and/or eliminating disease(s) of the nervous system in a subject. Examples of routes of administration which may be used include injection (subcutaneous, intravenous, parenterally, intraperitoneally, etc.), oral, inhalation, transdermal, and rectal. Depending on the route of administration, the creatine-like compound may be coated with or in a material to protect it from the natural conditions which may detrimentally effect its ability to perform its intended function. The administration of the creatine-like compound is done at dosages and for periods of time effective to reduce, ameliorate or eliminate the symptoms of the nervous system disorder. Dosage regimes may be adjusted for purposes of improving the therapeutic or prophylactic response of the compound. For example, several divided doses may be administered daily or the dose may be proportionally reduced as indicated by the exigencies of the therapeutic situation. Preferably, the composition is administered as a dietary supplement.

The dietary supplement may be added to typical feed carriers and binders, known in the animal feedstuffs industry. Exemplary carriers include cereal grains and grain or grass byproducts. The term “grain” includes such products as oats, barley, wheat, cannola, rye, sorgum, millet, corn, legumes, the latter including alfalfa and clover, and grasses including brome, timothy or fescue. Particularly preferred carriers include barley or other grains, grain or legume screenings, and oat groats. Often, an ingredient serves more than one purpose in the supplements of the present invention. For instance, if dextrose is used as an energy source in sufficient quantity, it also acts as a carrier. Similarly, molasses as an energy source also serves as a binder when combined with Molastix (Salsbury Laboratories Ltd., Kitchener, Ontario, Canada). Clays may also be used as binders.

In addition, the methods of the instant invention comprise creatine compounds effective in crossing the blood-brain barrier.

Utility

In the present invention, the creatine compounds can be administered to an individual (e.g., a mammal), alone or in combination with other compounds, for the prevention or treatment of TSEs diseases of the nervous system. As agents for the treatment of diseases of TSEs, creatine compounds can modify creatine kinase/phosphocreatine functions, energy state and cell survival thereby preventing, ameliorating, arresting or eliminating direct and/or indirect effects of disease which contribute to symptoms such as seen in BSE. Other compounds which can be administered together with the creatine compounds include drugs used for the treatment of neurodegenerative diseases or supplements which help preserve nervous system cells such as Q10 and vitamins, such as vitamine E. Also energy enhancing agents could be added such as pyruvate, nicotinamide, and camitine. The creatine compounds could be developed as supplements added to the diet of the mammals or as medical foods or as drugs.

A variety of TSE diseases can be prevented or treated with creatine or creatine analogs, including but not limited to BSE. Creatine or analogs of creatine can be used to prevent, reduce the severity of a disease, or reduce symptoms of primary disease episodes. Creatine, creatine phosphate or analogs such as creatine-pyruvate, creatine ascorbate, 3-guanidinopropionic acid and guanidino acetate can be used to treat progressive diseases. Many creatine analogs can cross the blood-brain barrier and hence can affect clinical symptoms.

Equivalents

Those skilled in the art will recognize, or be able to ascertain using no more than routine experimentation, many equivalents to the specific embodiments of the invention described herein. Such equivalents are intended to be encompassed by the following claims.

The entire contents of all articles, patents and patent applications mentioned herein are expressly incorporated herein by references. The entire contents of U.S. patent application Ser. No. 08/853,174 filed on May 7, 1997 entitled, “Use of Creatine or Creatine Analogs is also hereby incorporated herein by reference.

What is claimed is:

1. A method for treating a subject afflicted with Transmissible Spongiform Encephalopathies (TSEs) comprising administering to the subject an effective amount of creatine, creatine phosphate or a creatine compound or a salt thereof, such that said subject is treated for said TSE.

2. The method of claim 1 wherein the subject is a mammal.

3. The method of claim 2 wherein the subject is a human.

4. The method of claim 2 wherein the subject is cattle.

5. The method of claim 1 wherein said TSEs is scrapie.

6. The method of claim 1 wherein said TSEs is Bovine Spongiform Encephalopathy (BSE).

7. The method of claim 1 wherein said TSE is Creutzfeldt-Jakob disease (CJD).

8. A method for prevention or treatment of a subject afflicted with TSE comprising administering an effective amount of a creatine compound to said subject such that the subject is treated for TSE, wherein said creatine compound is of the general formula: 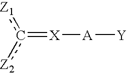
and pharmaceutically acceptable salts thereof, wherein:
a) Y is selected from the group consisting of: —CO₂H, —NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of: C, CH, C₁-C₅alkyl, C₂-C₅alkenyl, C₂-C₅alkynyl, and C₁-C₅ alkoyl chain, each having 0-2 substituents which are selected independently from the group consisting of:
1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
2) an aryl group selected from the group consisting of: a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy; and
3) —NH—M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:
1) hydrogen;
2) K where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
4) a C₅-C₉ a-amino-w-methyl-w-adenosylcarboxylic acid attached via the w-methyl carbon;
5) a C₅-C₉ a-amino-w-aza-w-methyl-w-adenosylcarboxylic acid attached via the w-methyl carbon; and
6) a C₅-C₉ a-amino-w-thia-w-methyl-w-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
1) hydrogen;
2) K, where K is selected from the group consisting of: C₁-C₆ straight alkyl; C₂-C₆ straight alkenyl, C₁-C₆ straight alkoyl, C₃-C₆ branched alkyl, C₃-C₆ branched alkenyl, and C₄-C₆ branched alkoyl, K having O-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
4) a C₄-C₈ a-amino-carboxylic acid attached via the w-carbon;
5) B, wherein B is selected from the group consisting of: —CO₂H, —NHOH, —SO₃H, —NO₂, OP(═O)(OH)(OJ) and —P(═O)(OH)(OJ), wherein J is selected from the group consisting of: hydrogen, C₁-C₆ straight alkyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
6) —D—E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of: —(PO₃)ₙNMP, where n is 0-2 and NMP is ribonucleotide monophosphate connected via the 5′-phosphate, 3′-phosphate or the aromatic ring of the base; —[P(═O)(OCH₃)(O)]ₘ—Q, where m is 0-3 and Q is a ribonucleoside connected via the ribose or the aromatic ring of the base; —[P(═O)(OH)(CH₂)]ₘ—Q, where m is 0-3 and Q is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
7) —E, wherein E is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ—Q, where m is 0-3 and Q is a ribonucleoside connected via the ribose or the aromatic ring of the base; —[P(═O)(OH)(CH₂)]ₘ—Q, where m is 0-3 and Q is a ribonucleoside connected via the ribose or the aromatic ring of the base; and an aryl group containing 0-3 substituents chose independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E is aryl, E may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

9. The method of claim 8, wherein said subject is cattle.

10. The method of claim 8, wherein said subject is human.

11. The method of claim 8 wherein the treatment comprises reducing or eliminating symptoms associated with a preexisting TSEs disease of the nervous system within the subject.

12. The method of claim 8 wherein the treatment comprises preventing the occurrence of TSEs diseases of the nervous system within the subject.

13. The method of claim 8 wherein the creatine compound is creatine in salt and hydrated forms.

14. The method of claim 8 wherein the creatine compound is creatine phosphate.

15. The method of claim 8 wherein the creatine compound is cyclocreatine.

16. The method of claim 8 wherein the creatine compound is cyclocreatine phosphate.

17. The method of claim 8 wherein the creatine compound is homocyclocreatine.

18. The method of claim 8 wherein the creatine compound is 3-guanidinopropionic acid.

19. The method of claim 8 wherein the creatine compound is guanidinoacetate.

20. The method of claim 8 wherein the creatine compound is creatine-pyruvate or creatine ascorbate.

21. The method of claim 8 further comprising coadministering to the subject an effective amount of an approved drug for the treatment of diseases of the nervous system.

22. The method of claim 8 further comprising coadministering to the subject an effective amount of a supplement that protects cells of the nervous system.

23. The method of claim 22, wherein said supplement is selected from the group consisting of vitamins, antioxidants, and energy enhancing agents.

24. The method of claim 8, wherein said creatine compound is administered orally.

25. The method of claim 8, wherein said creatine compound is administered as a food supplement.

26. The method of claim 8, wherein said effective amount is effective to prevent TSEs diseases.

27. The method of claim 25 wherein the creatine compound is creatine-pyruvate or creatine-ascorbate.

28. A method of claim 25 wherein the creatine compound is a guanidino benzoate.

29. A dietary supplement for the treatment or prevention of TSEs in a subject comprising an effective amount of a creatine, creatine phosphate, or a creatine compound or a salt thereof to treat or prevent TSEs in said subject.

30. The dietary supplement of claim 29, wherein said subject is cattle.

31. The dietary supplement of claim 29, wherein said TSE is BSE.

32. The dietary supplement of claim 29, wherein said dietary supplment comprises creatine.

33. The dietary supplement of claim 29, wherein the creatine compound is creatine phosphate.

34. The dietary supplement of claim 29, wherein the creatine compound is cyclocreatine.

35. The dietary supplement of claim 29, wherein the creatine compound is cyclocreatine phosphate.

36. The dietary supplement of claim 29, wherein the creatine compound is homocyclocreatine.

37. The dietary supplement of claim 29, wherein the creatine compound is 3-guanidinopropionic acid.

38. The dietary supplement of claim 29, wherein the creatine compound is guanidinoacetate.

39. The dietary supplement of claim 29, wherein the creatine compound is creatine-pyruvate or creatine ascorbate.
